(19) United States
(12) Patent Application Publication
(10) Pub. No.: US 2011/0091570 A1
(43) Pub. Date: Apr. 21, 2011

(54) Compositions and Devices for Antisepsis and Anticoagulation

(76) Inventor: Waldemar Gottardi, Innsbruck (AT)

(21) Appl. No.: 12/990,381

(22) PCT Filed: Mar. 17, 2009
(86) PCT No.: PCT/IB2009/000564

§ 371 Date: Oct. 29, 2010

Related U.S. Application Data
(60) Provisional application No. 61/045,058, filed on Apr. 15, 2008.

(51) Int. Cl. A61K 33/00; C07C 309/14; A61K 31/185; A61K 9/00; A61K 31/727; A61K 38/02; C07C 229/02; A61K 31/197; C07K 2/00; C07C 239/04; A61K 31/131; A61P 31/00; A61P 7/02; C01B 21/09

(52) U.S. Cl. 424/600; 562/104; 514/578; 424/400; 514/56; 514/2.3; 514/13.8; 562/574; 514/561; 530/350; 530/300; 564/118; 514/612; 423/413; 424/423

(57) ABSTRACT

Disclosed herein are compositions, methods, uses, and devices having antiseptic and anticoagulation properties in a mammal. The compositions, methods, uses, and devices are based on a therapeutically effective amount of one or more N-halogenated or N,N-dihalogenated amines, analogues or derivatives thereof, or pharmaceutically acceptable salts and esters. The preferred compound is N-chlorotaurine.

### FIELD OF THE INVENTION

The invention relates generally to pharmaceutical compositions and medical devices having antiseptic and anticoagulation capabilities. The invention relates particularly to N-halogenated or N,N-dihalogenated amine compositions, devices, methods, and uses having antiseptic and anticoagulation properties. Applicant incorporates by reference U.S. provisional application 61/021,823, filed Jan. 17, 2008, in its entirety.

### BACKGROUND OF THE INVENTION

Invasive medical devices (e.g., catheters) are widely used in human and veterinary medicine for many applications including, but not limited to, introduction of medications into the blood circulation. Invasive medical devices such a catheter can be fixed on the skin or implanted underneath the skin, sometimes penetrating a blood vessel. A common complication of such invasive medical devices is infection. The devices can become contaminated by bacteria, fungi, viruses, or other infective organisms or agents (e.g., proteins), resulting in systemic and/or localized infection. Similar concerns lie with other invasive medical devices, such as intubation tubes, surgical drains, and tracheostomy tubes.

One approach to preventing infection from catheters and other invasive devices is to fill their reservoirs with an antiseptic. There is, however, the possibility that small amounts of the antiseptic will be introduced into the blood circulation. In general, antiseptics can be toxic upon systemic application. Accordingly, they are not generally used for disinfecting in-dwelling catheters. For instance, chloramine T, a representative of the active chlorine compounds (chloramines), was applied intravenously to treat infected injuries in World War I. This treatment led to severe side effects such as pericardial and lung edema [Ref. 1]. Accordingly, there remains a need for improved antimicrobial compositions for use with a variety of invasive medical devices.

A second complication of invasive medical devices, such as catheters, is blood coagulation and obstruction of the device. Subsequent to injections or blood-taking via the catheter, it is irrigated with physiologic saline solution and then usually filled with the anticoagulant heparin. Thus, antimicrobial agents compatible with heparin would be of significant advantage for use with invasive medical devices.

Third, with rising concern as to hospital acquired infections or conditions, there is a need for new and improved compositions and methods for preventing and treating such infections and conditions. Examples of hospital acquired infections and conditions include, but are not limited to, catheter-associated urinary tract infections; pressure ulcers (e.g., decubitus ulcers); vascular catheter-associated infections; blood stream infections or septicaemia; infections caused by contamination at the surgical site (“surgical site infections”); and mediastinitis after coronary artery bypass graft surgery.

Also of pressing concern is the rise in antibiotic-resistant pathogens, such as methicillin resistant Staphylococcus aureus (MRSA); vancomycin-resistant Enterococci (VRE); penicillin-resistant Enterococcus; linezolid-resistant Enterococcus; vancomycin-resistant Staphylococcus aureus (VRSA) (also known as GISA (glycopeptides intermediate Staphylococcus aureus) or VISA (vancomycin insensitive Staphylococcus aureus)); and Acinetobacter baumannii. There is a need for new and improved compositions and methods for preventing and treating infections caused by antibiotic-resistant pathogens.

### SUMMARY OF THE INVENTION

Disclosed herein are pharmaceutical compositions with antiseptic and anti-infective activity coupled with anticoagulant activity for preventing and treating microbial infection as well as the formation of blood platelet aggregates, the formation of fibrin, thrombus formation and embolus formation in a mammal. The pharmaceutical compositions comprise a N-halogenated or N,N-dihalogenated amine (“haloamine”). For example, the N-halogenated or N,N-dihalogenated amines can be N-chlorotaurine (NCT) or a sodium salt thereof, in a pharmaceutically acceptable carrier. The compositions can include other anticoagulants (e.g., heparin), anti-platelet agents, and/or thrombolytic agents.

Also disclosed herein are medical devices in which the foregoing inventive compositions are incorporated into or coated onto the devices. The inventive devices include all devices that contact or penetrate the skin or a bodily organ, and all bodily implants, including, for example, devices used in blood collection, blood circulation, and blood storage, such as catheters, Port-A-Cath® catheters, blood dialysis machines, blood collection syringes, tubes, blood lines, urinary tract catheters, central line catheters, central venous catheters, IV drip units, implantable catheters, shunts, stents, and implants of all sorts.

Also disclosed herein are methods of inhibiting infection and coagulation in mammals, including treating a mammal in need of such treatment with a pharmaceutically effective dose of the inventive compositions and/or using one or more of the inventive devices to both inhibit infection and coagulation. Such methods include, but are not limited to methods for prevention of infection and blood coagulation within catheters (e.g., Port-A-Cath® catheters, urinary tract, central line, and central venous catheters); treating urinary disorders; performing hemodialysis; providing artificial shunts, joints and other artificial structures; treating or preventing myocardial infarction, unstable angina, stroke, restenosis, deep vein thrombosis, disseminated intravascular coagulation caused by trauma, sepsis or tumor metastasis, cardiopulmonary bypass surgery, hypercoagulability during chemotherapy, fibrin formation in the eye, and wound healing.

Also disclosed herein are compositions and methods for preventing and treating hospital-acquired infections and conditions, wherein a mammal in need of such treatment is provided with a pharmaceutically effective dose of an inventive composition and/or an inventive device to inhibit infection. Examples of hospital acquired infections and conditions include, for example, catheter-associated urinary tract infections; pressure ulcers (e.g., decubitus ulcers); vascular catheter-associated infections; blood stream infections or septicaemia; infections caused by contamination at the surgical site (“surgical site infections”); and mediastinitis after coronary artery bypass graft surgery.

Also disclosed herein are compositions and methods for preventing and treating antibiotic-resistant pathogens, such as methicillin resistant Staphylococcus aureus (MRSA); vancomycin-resistant Enterococci (VRE); penicillin-resistant Enterococcus; linezolid-resistant Enterococcus; vancomycin-resistant Staphylococcus aureus (VRSA) (also known as GISA (glycopeptides intermediate Staphylococcus aureus) or VISA (vancomycin insensitive Staphylococcus aureus)); and Acinetobacter baumannii.

Also disclosed herein are uses of one or more N-halogenated or N,N-dihalogenated amines for the manufacture of medicaments having antiseptic and anticoagulation capabilities. The medicaments can be used in any of the compositions, devices, and/or methods described herein,

In various embodiments of the invention, including any of the compositions, devices, methods, and/or uses described herein, the composition includes a therapeutically effective amount of one or more N-halogenated or N,N-dihalogenated amines. As used herein, the terms “N-halogenated amine(s)” and “N,N-dihalogenated amine(s)” include analogues and derivatives thereof, and pharmaceutically acceptable salts or esters of any of the foregoing compounds. In various preferred embodiments of the invention, the halogen of the N-halogenated or N,N-dihalogenated amine is chlorine. In other embodiments, the halogen can be any group 17 element such as fluorine, bromine, or iodine.

In various embodiments of the invention, including any of the compositions, devices, methods, and/or uses described herein, a N-halogenated or N,N-dihalogenated amine can be a derivative of a protein, peptide, or amino acid, or pharmaceutically acceptable salts thereof. Additionally, the amine can be derived from at least one of an α-amino carbonic acid (e.g., glycine, alanine, leucine), a β-amino carbonic acid (e.g., β-alanine), an α-amino sulfonic acid (e.g., aminomethane sulfonic acid), a β-amino sulfonic acid (e.g., taurine and its derivatives alkylated at a carbon, e.g., dimethyltaurine), and an aliphatic amine (e.g., ethylamine). In certain preferred embodiments, the N-halogenated or N,N-dihalogenated amine is N-chlorotaurine (NCT) or N,N-dichlorotaurine (NDCT), or pharmaceutically acceptable salts or esters thereof. NCT is a particularly preferred embodiment.

In some embodiments of the invention, including any of the compositions, devices, methods, and/or uses described herein, the composition comprises an analogue or derivative of N-halogenated or N,N-dihalogenated amine, e.g., alkylated derivatives such as N,N-dichloro-2,2-dimethyl taurine.

In certain embodiments of the invention, including any of the compositions, devices, methods, and/or uses described herein, the N-halogenated or N,N-dihalogenated amine can be in the form of an alkali salt; preferably a sodium salt.

Compositions of the invention, including any of the compositions, devices, methods, and/or uses described herein, can comprise one or more N-halogenated or N,N-dihalogenated amines at a concentration of about 0.001% to about 10% weight per volume. In various preferred embodiments a N-halogenated or N,N-dihalogenated amine is present at a concentration of about 0.01% to about 5%; about 0.01% to about 2%; about 0.1% to about 2%; about 0.2% to about 1%; about 0.01% to about 0.035%; or about 0.01% to about 0.025%.

In certain preferred embodiments of the invention, the compositions comprise one or more N-halogenated or N,N-dihalogenated amines in combination with an ammonium salt; preferably ammonium chloride. Particularly preferred is a combination of NCT and/or NDCT, or their alkylated derivatives, with ammonium chloride. Preferably, each of the constituents of the combined composition (i.e., the one or more N-halogenated or N,N-dihalogenated amines and the ammonium salt) can be at a concentration of about 0.02% to about 1%, more preferably about 0.1% to about 0.5%. A preferred ratio for the one or more N-halogenated and N,N-dihalogenated amines to the ammonium salt is about 1:1. For some indications, the ratio of the one or more N-halogenated or N,N-dihalogenated amines to the ammonium salt is preferably about 1:0.1.

It is understood that any aspect, feature, or embodiment of the invention, whether characterized as preferred or not characterized as preferred, can be combined with any other aspect, feature or embodiment of the invention, without departing from the scope of the invention. Other aspects and advantages of the invention will become apparent from the following drawings and description, all of which illustrate principles of the invention, by way of example only.

### BRIEF DESCRIPTION OF THE DRAWINGS

The advantages of the invention described above, together with further advantages, may be better understood by referring to the following description taken in conjunction with the accompanying drawings.

FIG. 1 presents a plot of bactericidal activity of 1% NCT without and with 125 IE/mL heparin against Staphylococcus aureus ATCC 25923 at pH 7.1 and 37° C. Mean values standard error of the mean of three independent experiments. P>0.05 between NCT without and NCT with heparin.

FIG. 2 presents a plot of bactericidal activity of 1% NCT without and with 125 IE/mL heparin against Escherichia coli ATCC 11229 at pH 7.1 and 37° C. Mean values±standard error of the mean of three independent experiments. P>0.05 between NCT without and NCT with heparin.

FIG. 3 presents a plot of bactericidal activity of 1% NCT without and with 125 IE/mL heparin against Streptococcus pyogenes at pH 7.1 and 37° C. Mean values±standard error of the mean of three independent experiments. P>0.05 between NCT without and NCT with heparin.

FIG. 4 presents a plot of bactericidal activity of 1% NCT without and with 125 IE/mL heparin against Staphylococcus epidermidis at pH 7.1 and 37° C. Mean values standard error of the mean of three independent experiments. P>0.05 between NCT without and NCT with heparin.

FIG. 5 presents a plot of bactericidal activity of 1% NCT without and with 125 IE/mL heparin against Pseudomonas aeruginosa at pH 7.1 and 37° C. Mean values±standard error of the mean of three independent experiments. P>0.05 between NCT without and NCT with heparin.

FIG. 6 presents a plot of bactericidal activity of 1% NCT without and with 125 IE/mL heparin against Proteus mirabilis at pH 7.1 and 37° C. Mean values±standard error of the mean of three independent experiments. P>0.05 between NCT without and NCT with heparin.

FIG. 7 presents a plot of bactericidal activity of 1% NCT without and with 125 IE/mL heparin against Methicillin resistant Staphylococcus aureus (MRSA) (clinical isolate 509) at pH 7.1 and 37° C. Mean values±standard error of the mean of three independent experiments. P>0.05 between NCT without and NCT with heparin.

FIG. 8 presents a plot of bactericidal activity of 1% NCT without and with 125 IE/mL heparin against Methicillin resistant Staphylococcus aureus (MRSA) (clinical isolate 435) at pH 7.1 and 37° C. Mean values±standard error of the mean of three independent experiments. P>0.05 between NCT without and NCT with heparin.

FIG. 9 presents a plot of fungicidal activity of 1% NCT without and with 125 IE/mL heparin against Candida albicans (CBS 5982) at pH 7.1 and 37° C. Mean values±standard error of the mean of three independent experiments. P>0.05 between NCT without and NCT with heparin.

FIG. 10 presents a plot of bactericidal activity of 1% NCT with 125 IE/mL heparin in human blood against Staphylococcus aureus (ATCC 25923) at pFI 7.1 and 37° C. Mean values±standard error of the mean of three independent experiments. P<0.01 between NCT and control samples.

FIG. 11 presents a plot of bactericidal activity of 1% NCT with 125 IE/mL heparin in human blood against Escherichia coli (ATCC 11229) at pH 7.1 and 37° C. Mean values±standard error of the mean of three independent experiments. P<0.01 between NCT and control samples.

### DETAILED DESCRIPTION OF THE INVENTION

The invention, in all of its various aspects and embodiments, comprises one or more N-halogenated and N,N-dihalogenated amines in compositions, devices, methods, and/or uses having antiseptic and anticoagulation properties in mammals. As used herein, the terms “N-halogenated amine(s)” and “N,N-dihalogenated amine(s)” include analogues and derivatives thereof, and pharmaceutically acceptable salts or esters of any of the foregoing compositions, devices, methods, and/or uses. Preferred derivatives include alkylated derivatives such as N,N-dichloro-2,2-dimethyl taurine. Suitable salts can be prepared by known methods, including but not limited to the method described in German Patent Application 4041703 by Gottardi (incorporated by reference herein in its entirety). Sodium and potassium salts are preferred; sodium salts are particularly preferred.

The halogen of the N-halogenated and N,N-dihalogenated amines may be any group 17 element, such as fluorine, bromine, or iodine; preferably the halogen is chlorine. The amine of the N-halogenated and N,N-dihalogenated amines may be any amine including, e.g., a protein, peptide, or amino acid. Additionally, the amine can be derived from at least one of an α-amino carbonic acid (e.g., glycine, alanine, leucine), a β-amino carbonic acid (e.g., β-alanine), an α-amino sulfonic acid (e.g., aminomethane sulfonic acid), a β-amino sulfonic acid (e.g., taurine and its derivatives alkylated at a carbon, e.g., dimethyltaurine), and an aliphatic amine (e.g., ethylamine). Preferably, the amine is taurine.

In certain preferred embodiments of the invention, the one or more N-halogenated or N,N-dihalogenated amines are N-chlorotaurine (NCT) or N,N-dichlorotaurine (NDCT), or pharmaceutically acceptable salts or esters thereof. NCT and its sodium salt are particularly preferred embodiments.

As a particularly preferred embodiment of the invention, compositions, devices, methods, and/or uses comprising one or more N-halogenated and N,N-dihalogenated amines can be enhanced by the addition of an ammonium salt; preferably ammonium chloride. This can lead to formation of monochloramine in equilibrium which is lipophilic and penetrates pathogens better than the haloamine alone [Refs. 2, 3]. Each of these references is incorporated by reference in its entirety. Particularly preferred compositions and methods are those in which NCT and/or NDCT, or their alkylated derivatives, are combined with ammonium chloride.

N-halogenated and N,N-dihalogenated amines can be synthesized by known techniques. For example, NCT can be synthesized as a crystalline sodium salt in aqueous solution [Ref. 4]. Similarly, NDCT can be synthesized as described in Refs. 5 and 6. Formulations of NCT and ammonium chloride may be synthesized as disclosed in Ref. 7. Each of the foregoing references are herein incorporated by reference in their entirety.

In addition to the pharmacologically active compounds, the new pharmaceutical preparations can contain suitable pharmaceutically acceptable carriers comprising excipients and auxiliaries that facilitate processing of the active compounds into preparations that can be used pharmaceutically. In various embodiments, the compositions can include pharmaceutically acceptable carriers, such as buffers, stabilizers, solvents, preserving agents, diluents, extenders and other recognized auxiliary substances or excipients.

In various embodiments, the compositions can include other anticoagulants (e.g., heparin), anti-platelet agents, and/or thrombolytic agents.

In the compositions, devices, methods, and/or uses of the invention, the one or more N-halogenated and N,N-dihalogenated amines are present or administered at a concentration of about 0.001% to about 10% weight per volume. In various preferred embodiments a N-halogenated or N,N-dihalogenated amine is present at a concentration of about 0.01% to about 5%; about 0.01% to about 2%; about 0.1% to about 2%; about 0.2% to about 1%; about 0.01% to about 0.035%; or about 0.01% to about 0.025%.

When used for the purpose of sanitizing open surgical sites, an aqueous solution of haloamine may be applied to the site by local irrigation at a concentration and duration effective to decrease the risk of infection after surgery. A preferred haloamine for such applications is NCT. The haloamine may be applied in a concentration range of about 0.001% to about 10% of the active compound in an aqueous solution. Preferably, the active concentration range is about 0.01% to about 5%; more preferably about 0.1% to about 2%; and still more preferably about 0.2% to about 1%.

The compositions, devices, methods, and/or uses of the invention may include one or more N-halogenated or N,N-dihalogenated amines in combination with an ammonium salt; preferably ammonium chloride. Particularly preferred are compositions, devices, methods, and/or uses in which NCT and/or NDCT, or their alkylated derivatives, are combined with ammonium chloride. Preferably, each of the constituents of the combined composition (i.e., the one or more N-halogenated or N,N-dihalogenated amines and the ammonium salt) are administered at a concentration of about 0.02% to about 1%, more preferably about 0.1% to about 0.5%. A preferred ratio for the one or more N-halogenated and N,N-dihalogenated amines to the ammonium salt is about 1:1. For some indications, the ratio of the one or more N-halogenated or N,N-dihalogenated amines to the ammonium salt is preferably about 1:0.1.

Activity and Tolerability of Haloamines (e.g., NCT)

One exemplary haloamine is NCT, the N-chloro derivative of the amino acid taurine, which is a long-lived oxidant produced by human leukocytes during inflammation [Ref. 8]. NCT can save the oxidation capacity of originally formed hypochlorous acid, which can be detoxified by reaction with taurine (HOCl+taurine→N-chlorotaurine+H₂O) [Ref. 9]. In addition, NCT can downregulate proinflammatory cytokines and therefore it may be involved in termination of inflammation [Refs. 10-12].

NCT and other N-chloramines have broad-spectrum antimicrobial activity including representatives of all classes of pathogens [Refs. 2a, 2 and 22-25] and may contribute to inactivation of pathogens in vivo [Ref. 13]. Because of the unspecific oxidizing mechanism of reaction, resistance of pathogens is not induced by treatment with NCT [Ref. 2]. See also Ref. 26.

Furthermore, certain natural N-chloramines do not decompose to toxic compounds (above all NCT and other N-haloamines), and no signs for systemic resorption have been observed in the above mentioned clinical studies. N-chloramines react with reducing agents according to R2-N—Cl+H++2e-→R2-NH+Cl— (e.g., NCT converts to the endogenous products taurine and chloride by the reaction: ClHN—CH₂—CH₂—SO₃-+2H++2e-→H₃N+—CH₂—CH₂—SO₃—+Cl—). The absence of residues and decay products can be a general advantage of haloamines compared to other antimicrobial agents.

NCT is relatively safe and well-tolerated by mammals in topical and localized applications. This was demonstrated in rabbit and human eyes [Ref. 13]. Also, data indicating efficacy in infectious conjunctivitis are available [Ref. 14]. In human external otitis, NCT was more effective than a standard medication [Ref 15]. A pilot study in chronic rhinosinusitis demonstrated good tolerability [Ref. 16]. Treatment of purulent coated crural ulcers with NCT caused significantly less pain and was less toxic than chloramine T, the standard for decades in our University hospital [Ref. 17]. It is possible to eradicate bacteria from the urinary bladder with NCT irrigations as shown in three patients suffering from inflammation with omniresistant Pseudomonas aeruginosa [Ref 18]. Furthermore, transtympanal injection of 0.1%, 1%, and 10% NCT to the middle ear of mice did not cause damage of the inner ear [Ref. 19]. The same was true for guinea pigs where 10 μL of 1% and 0.1% were injected repeatedly to the middle ear via an implanted catheter system [Ref 20]. Local administration of NCT inhibited septic arthritis by Staphylococcus aureus in a mouse model [Ref 21]. In a further study, Swiss mice tolerated up to one mL 1% NCT upon intraperitoneal injection.

Surprisingly, NCT is tolerated by mice even when injected intravenously into the tail vein. In this model, injection of 1% aqueous NCT solution was tolerated at a volume that equals approximately 10% of the total blood volume of a mouse. A 1% solution contains 55 mmol/L NCT, which is more than 1000 times greater than any physiologic concentration produced by human leukocytes.

Chloramines are inactivated by reaction with sulphur-containing molecules (thio groups), a phenomenon known as “chlorine-consumption” [Ref 4]. Thus, chloramines would be expected to lose activity in the presence of blood. However, the Examples below demonstrate that a 1% aqueous solution of NCT still has good microbicidal activity when the solution contains 75% whole blood.

Haloamines (e.g., NCT) as an Anticoagulant and Compatibility with Other Anticoagulants

We tested the antimicrobial activity of NCT in the presence of heparin, and discovered that the antimicrobial activity was not impaired. Vice versa, we discovered that the anticoagulant effect of heparin was not impaired by NCT. In addition, we discovered that NCT alone, without additives, has an anticoagulant effect. Anticoagulant effects of haloamines have been described by Stief et al. [Ref 27], who reported an anticoagulant effect of NCT at a concentration or 2-3 mmol/L, but not below. In the Examples below, there is demonstrated a significant anticoagulant effect of NCT at a concentration of 0.55 mmol/L, and a strong effect at 1.375 mmol/L. Prothrombin time, activated partial thromboplastin time, and thrombin time were prolonged, and fibrinogen decreased.

Administration and Applications

The pharmaceutical compositions of the invention can be administered to any mammal that can experience the beneficial effects of the compounds of the invention. Foremost among such mammals are humans, although the invention is not intended to be so limited.

The pharmaceutical compositions of the invention can be administered by any means that achieve their intended purpose. For example, administration can be by parenteral, subcutaneous, intravenous, intramuscular, intraperitoneal, transdermal, buccal, or ocular routes. The dosage administered will be dependent upon the age, health, and weight of the recipient, kind of concurrent treatment, if any, frequency of treatment, and the nature of the effect desired.

With dual antimicrobial and anticoagulant activities, the compositions disclosed herein can be useful for the treatment or prophylaxis of states characterized by antimicrobial infection, abnormal or hyperactive coagulation, or a combination of antimicrobial infection and abnormal or hyperactive coagulation. These states include, but are not limited to, urinary disorders; hemodialysis; deep vein thrombosis; disseminated intravascular coagulopathy, which occurs during septic shock; myocardial infarction; stroke; coronary artery bypass; fibrin formation in the eye; hip replacement; and thrombus formation resulting from either thrombolytic therapy or percutaneous transluminal coronary angioplasty (PCTA).

The inventive compositions may be dispensed directly to the patient, or incorporated into or coated onto medical devices that can be introduced into patients, especially devices that are used in patients for long periods of time. For example, the inventive compositions may be used to fill the reservoir of totally implantable venous access systems, such as the Port-a-Cath® by Smiths Medical MD, Inc., and similar devices. In other embodiments, the inventive compositions may be coated onto a device, or incorporated into gel, polymer, or foam device coatings. The inventive compositions can be especially useful when incorporated into or onto devices used in blood collection, blood circulation, and blood storage, such as catheters, blood dialysis machines, blood collection syringes and tubes, and blood lines. The inventive compositions may also be used as an anticoagulant in extracorporeal blood circuits.

Metal stents have been shown to reduce restenosis, but can be thrombogenic. A strategy for reducing the thrombogenicity of stents is to coat, embed, adsorb or covalently attach a thrombin-inhibiting agent to the stent surface. The inventive compositions can be employed for this purpose. For example, the compositions can be attached to, or embedded within soluble and/or biodegradable polymers and thereafter coated onto stent materials. Such polymers can include polyvinylpyrrolidone, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

By virtue of the effects of thrombin on a host of cell types, such as smooth muscle cells, endothelial cells and neutrophils, the inventive compositions may additionally be used in the treatment or prophylaxis of adult respiratory distress syndrome; inflammatory responses; wound healing; reperfusion damage; atherosclerosis; and restenosis following an injury such as balloon angioplasty, atherectomy, and arterial stent placement.

When employed as inhibitors of thrombin, the inventive compositions may be used in combination with thrombolytic agents such as tissue plasminogen activator, streptokinase, and urokinase. Additionally, the compounds of the invention may be used in combination with other antithrombotic or anticoagulant drugs such as, but not limited to, fibrinogen antagonists and thromboxane receptor antagonists.

The inventive compositions may also be coupled with soluble polymers as targetable drug carriers. Such polymers can include, for example, polyvinylpyrrolidone and pyran copolymer. Furthermore, the inventive compositions may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogens.

### Example 1

Balb/c mice were injected into the tail vein with a sterile aqueous solution of 1% NCT. Four mice were injected with 0.05 ml, four mice with 0.1 ml, one mouse with 0.2 ml, and one mouse with 0.3 ml, respectively, of the NCT solution. There were no changes of behaviour, and all mice survived.

In this Example, injection of 1% aqueous NCT solution was tolerated in the mouse model at a volume that equals approximately 10% of the total blood volume of a mouse.

### Example 2

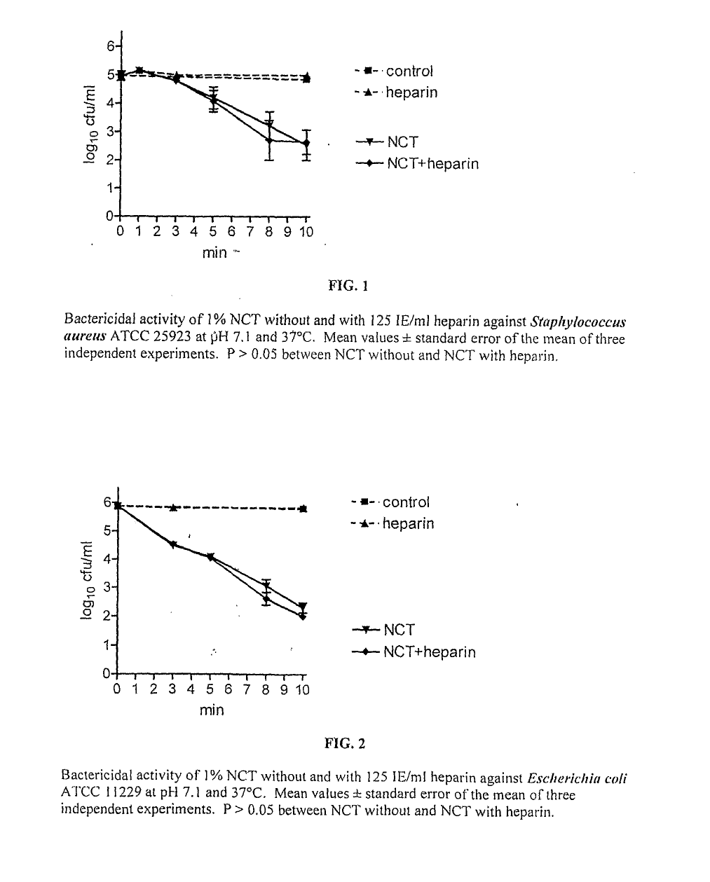

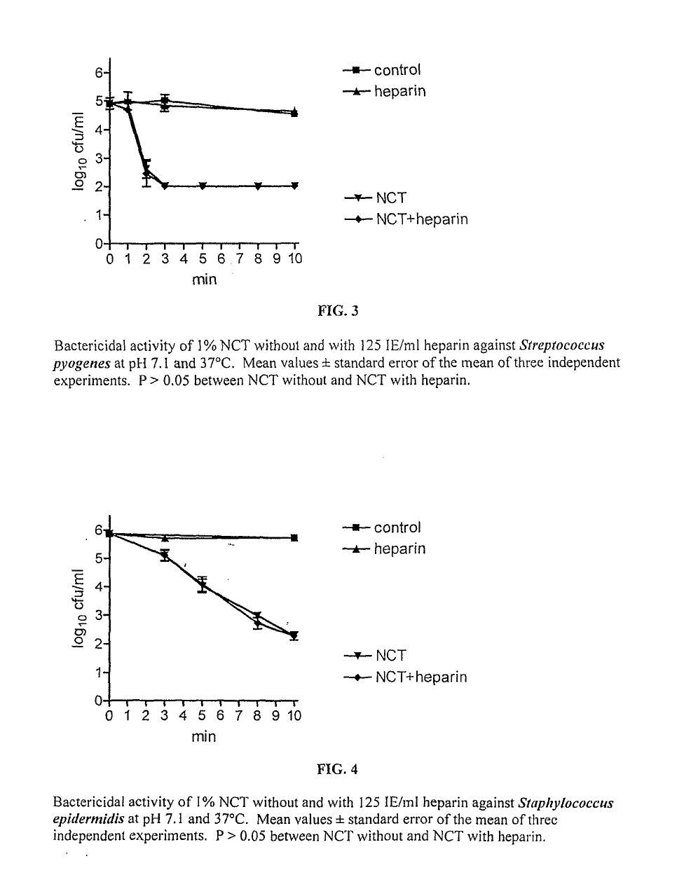

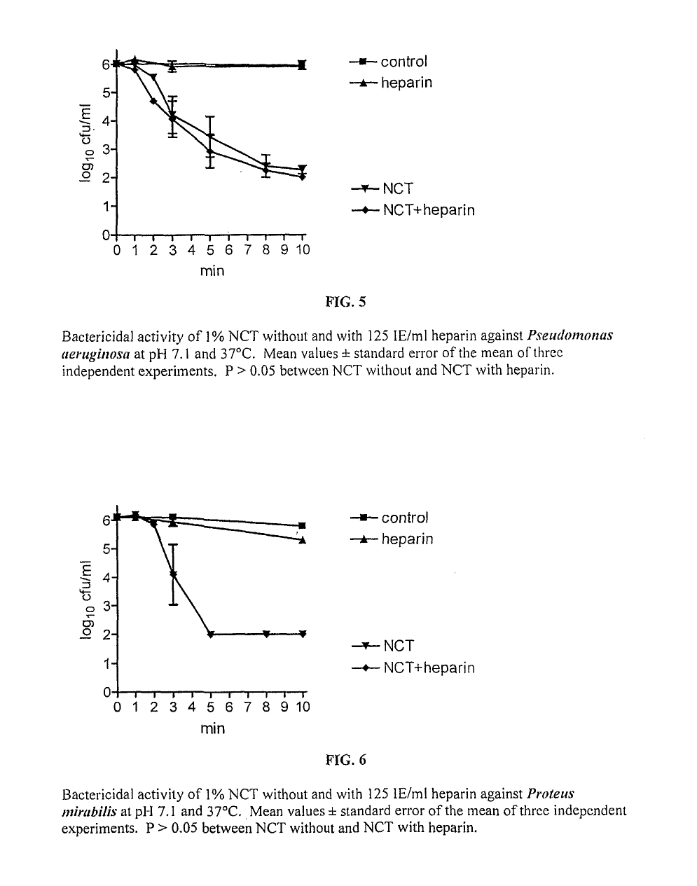

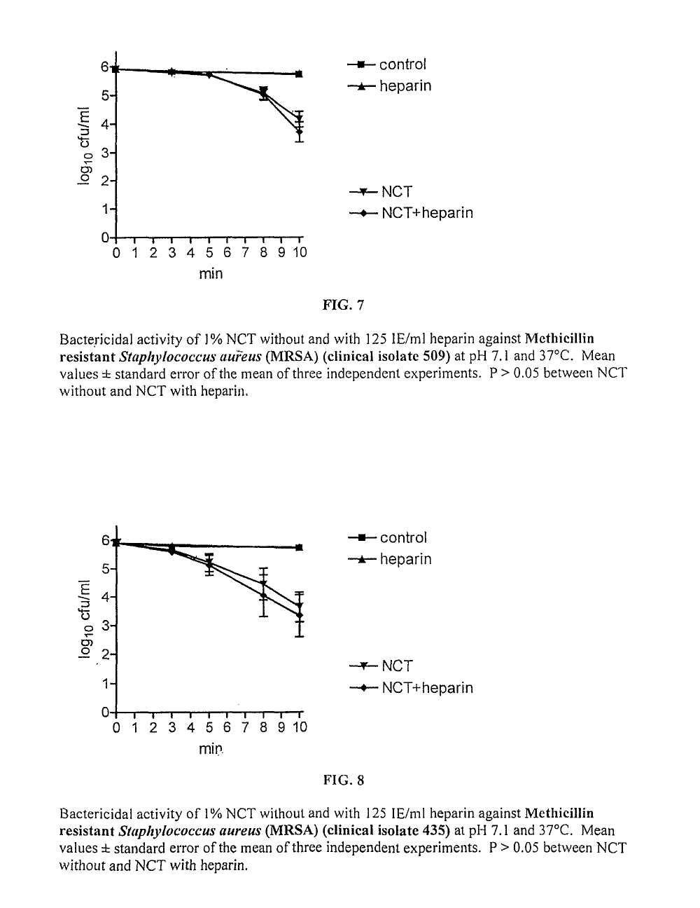

NCT was dissolved in 0.1 M phosphate buffer containing either 125 IE/mL heparin or no heparin. The final NCT concentration was 1%. Various bacterial species were grown in tryptic soy broth overnight, were washed twice in saline and then suspended in the NCT and NCT/heparin solutions. The species were Staphylococcus aureus (ATCC 25923), Escherichia coli (ATCC 11229), Streptococcus pyogenes, Staphylococcus epidermidis, Pseudomonas aeruginosa, Proteus mirabilis, and methicillin-resistant Staphylococcus aureus. Controls were performed with the foregoing bacterial species suspended in 0.1 M phosphate buffer without additives and in 0.1 M phosphate buffer containing 125 IE/mL heparin. All of the solutions were incubated at 37° C. and pH 7.1. After 1, 3, 5, 8, and 10 minutes, aliquots of 100 μL were removed and diluted 10-fold or 100-fold in 0.6% sodium thiosulfate solution to inactivate the NCT. Aliquots (50 μL) of these dilutions were spread on tryptic soy agar plates. The plates were incubated at 37° C. and colony forming units of bacteria were counted after 24 and 48 hours. The assays were performed three times serially and the mean count was reported. (FIGS. 1-8). There was no difference in the killing activity of NCT with or without heparin. Statistical analysis was performed by Student's paired t test and one way analysis of variance (P>0.05 between NCT without heparin and NCT with heparin). This means that the microbicidal activity of NCT was not impaired by heparin. Additionally, virtually identical experiments run with 0.1% NCT revealed no statistically significant influence of heparin on the killing of bacteria.

### Example 3

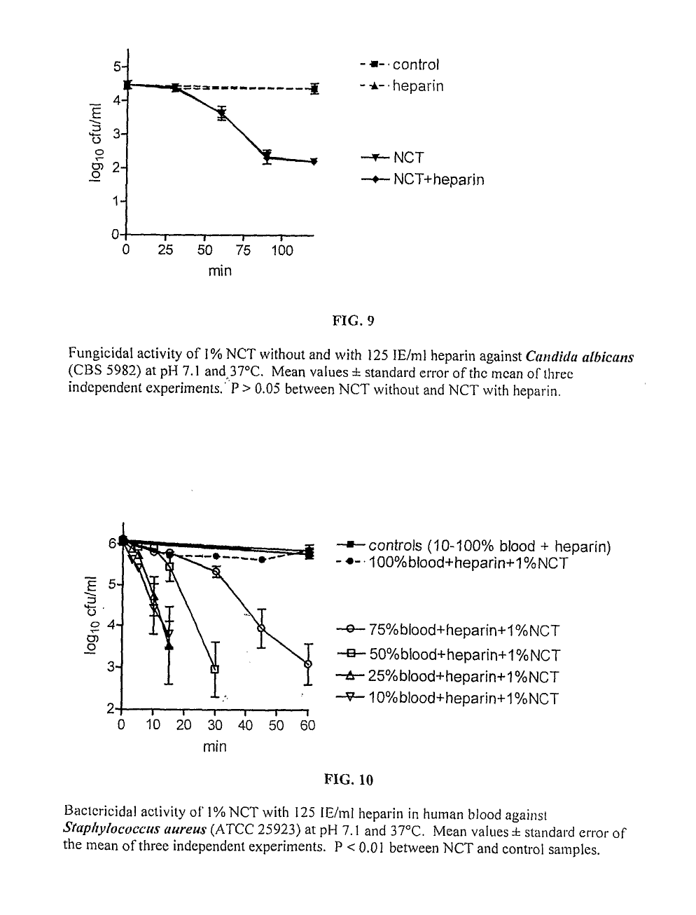

NCT was dissolved in 0.1 M phosphate buffer containing either 125 IE/mL heparin or no heparin. The final NCT concentration was 1%. Yeast (Candida albicans, CBS 5982) grown in tryptic soy broth overnight was washed twice in saline and then suspended in the NCT and NCT/heparin solutions. Controls were performed with the yeast suspended in 0.1 M phosphate buffer without additives and in 0.1 M phosphate buffer containing 125 IE/mL heparin. After incubation times of 30, 60, 90 and 120 minutes at 37° C. and pH 7.1, aliquots of 100 μL were removed and diluted tenfold or 100-fold in 0.6% sodium thiosulfate solution to inactivate the NCT. Aliquots (50 μL) of these dilutions were spread on tryptic soy agar plates. The plates were incubated at 37° C. and colony forming units of Candida albicans were counted after 24 and 48 hours. The assays were performed three times serially and the mean count was reported (FIG. 9). There was no statistically significant difference in the killing activity of NCT without or with heparin. Statistical analysis was performed by Student's paired t test and one way analysis of variance (P>0.05 between NCT without and NCT with heparin). This means that the microbicidal activity of NCT was not impaired by heparin.

### Example 4

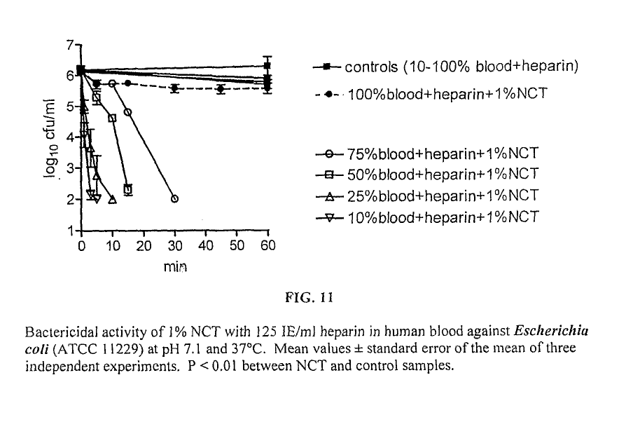

Bacteria (Staphylococcus aureus, ATCC 25923 and Escherichia coli, ATCC 11229) grown in tryptic soy broth overnight were washed twice in saline and suspended in human whole blood containing 125 IE/mL heparin. Different volumes of a saline solution of NCT sodium salt were added to the suspension so that the test samples contained 10%, 25%, 50%, and 75% blood. In addition, 100% blood plus 125 IE/mL heparin was tested at which NCT was directly dissolved in blood. The final NCT concentration after mixing blood and NCT solutions was 1% in all samples. Controls contained 100% blood plus 125 IE/mL heparin and 10%, 25%, 50% and 75% blood, respectively, in saline, plus 125 IE/mL heparin without NCT. All of the solutions were incubated at 37° C. and pH 7.36-7.44 (the pH of blood). After 1, 3, 5, 10, 15, 30, 45 and 60 minutes, aliquots of 100 μL were removed and diluted tenfold or 100-fold in 0.6% sodium thiosulfate solution to inactivate the NCT. Aliquots (50 μL) of these dilutions were spread on tryptic soy agar plates. The plates were incubated at 37° C. and colony forming units of bacteria were counted after 24 and 48 hours. The assays were performed three times serially and the mean count was reported (FIGS. 10 and 11). NCT demonstrated bactericidal activity in 10%, 25%, 50% and 75% blood plus 125 IE/mL heparin. As we expected, when NCT was dissolved in 100% blood, no significant bactericidal activity was detected. Statistical analysis was performed by Student's paired t test and one way analysis of variance (P>0.05 between NCT without and NCT with heparin).

### Example 5

Whole blood was taken from volunteers and enriched with trisodium citrate (0.3 mL of a 0.106 molar solution per 3 mL whole blood) (S-Monovette®, Sarstedt, Nümbrecht, Germany). Routine coagulation tests were performed on the following samples:

1.8 mL citrate blood plus 0.2 mL 0.9% saline;

1.8 mL citrate blood plus 0.25 IE heparin plus 0.2 mL 0.9% saline;

1.8 mL citrate blood plus 0.25 IE heparin plus 0.2 mL 10% NCT in 0.9% saline; and

1.8 mL citrate blood plus 0.2 mL 10% NCT in 0.9% saline.

The following commercial coagulation tests were performed according to the prescription of the manufacturer: prothrombin time (PT) was determined using Thromborel® S test (Dade Behring GmbH, Marburg, Germany); activated partial thromboplastin time (aPTT) was determined using Pathromtin* SL test (Dade Behring GmbH, Marburg, Germany); and thrombin time (TT) was determined using BC Thrombin Reagent test (Dade Behring GmbH, Marburg, Germany).

Sample (A) showed normal coagulation, while with Sample (B) the activated partial thromboplastin time increased from 36 to 56 seconds. With Sample (C), prothrombin time, activated partial thromboplastin time, and thrombin time were prolonged above the test limits. Additionally, Sample (D) revealed prolonged prothrombin time, activated partial thromboplastin time, and thrombin time above the test limits.

These results demonstrate that 1% NCT (which was the final concentration in the test solutions) does not counteract the anticoagulant activity of heparin. Furthermore, NCT alone (i.e., without heparin) has anticoagulant activity.

### Example 6

Whole blood was taken from a single volunteer and enriched with trisodium citrate (0.3 mL of a 0.106 molar solution per 3 mL whole blood) (S-Monovette®, Sarstedt, Nümbrecht, Germany). Portions of 1.8 mL citrate blood were mixed with 0.2 mL NCT in 0.9% saline. The following final (after dilution in blood) NCT concentrations were tested separately: 1% (55 mM); 0.5% (27.5 mM); 0.25% (13.75 mM); 0.1% (5.5 mM); 0.05% (2.75 mM); 0.025% (1.375 mM); and 0.01% (0.55 mM). A control without NCT was performed in parallel. The following commercial coagulation tests were performed according to the prescription of the manufacturer: prothrombin time (PT) was determined using Thromborel® S test (Dade Behring GmbH, Marburg, Germany), activated partial thromboplastin time (aPTT) was determined using Pathromtin* SL test (Dade Behring GmbH, Marburg, Germany); thrombin time (TT) was determined using BC Thrombin Reagent test (Dade Behring GmbH, Marburg, Germany); and fibrinogen was determined using Multifibren* U test (Dade Behring GmbH, Marburg, Germany).

Prothrombin time, activated partial thromboplastin time, and thrombin time were prolonged; and fibrinogen decreased (Table 1). The lowest concentrations showed small effects in the PT, aPTT, and fibrinogen tests. All concentrations exceeding 0.01% revealed increasing effects with increasing concentration (Table 1).

TABLE 1

| NCT Concentration | PT (%) | aPTT (sec) | TT (sec) | Fibrinogen |
|---|---|---|---|---|
| 1% | <limit | >limit | >limit | not detectable |
| 0.5% | <limit | >limit | >limit | not detectable |
| 0.25% | <limit | >limit | 101.8 | not detectable |
| 0.1% | <limit | 371.4 | 92.2 | not detectable |
| 0.05% | 33.4 | 71.3 | 48.8 | not detectable |
| 0.025% | 70.6 | 37.7 | 26.6 | 199.4 |
| 0.01% | 91.4 | 30.6 | 19.5 | 281.4 |
| no NCT | 100.1 | 29.1 | 19.6 | 388.3 |

Legend:
PT = prothrombin time
aPTT = activated partial thromboplastin time
TT = thrombin time
>limit = value exceeds the test limit

### Example 7

Whole blood was taken from 11 volunteers and enriched with trisodium citrate (0.3 mL of a 0.106 molar solution per 3 mL whole blood) (S-Monovette®, Sarstedt, Nümbrecht, Germany). Commercial coagulation tests (as described in Example 6) were performed with the following samples:

1.8 mL citrate blood plus 0.2 mL 0.9% saline;

1.8 mL citrate blood plus 0.25 IE heparin plus 0.2 mL 0.9% saline;

1.8 mL citrate blood plus 0.2 mL 0.1% NCT in 0.9% saline;

1.8 mL citrate blood plus 0.2 mL 0.25% NCT in 0.9% saline; and

1.8 mL citrate blood plus 0.25 IE heparin plus 0.2 mL 0.1% NCT in 0.9% saline;

The final concentration of NCT was 0.01% (0.55 mM) in samples (C) and (D), and 0.025% (1.375 mM) in sample (E).

Results are shown in Tables 2-8.

Prothrombin time, activated partial thromboplastin time, and thrombin time were prolonged; and fibrinogen decreased in samples containing NCT (C, D and E). The effects were markedly pronounced with 0.025% NCT, and less pronounced with 0.01% NCT, but also highly significant compared to the control (A).

These results clearly show a highly significant anticoagulant effect of NCT, which is independent of heparin. The effects of NCT and heparin do not necessarily impair each other but they can be additive.

TABLE 2
Activated partial thromboplastin time (aPTT) (sec)

| Subject no. | Control | Heparin | 0.01% NCT | 0.025% NCT | 0.01% NCT + Heparin |
|---|---|---|---|---|---|
| 1 | 31 | 400 | 35 | 91 | 400 |
| 2 | 35 | 216 | 40 | 167 | 329 |
| 3 | 33 | 284 | 37 | 70 | 301 |
| 4 | 32 | 195 | 36 | 91 | 260 |
| 5 | 28 | 169 | 31 | 71 | 185 |
| 6 | 34 | 283 | 39 | 93 | 343 |
| 7 | 36 | 187 | 41 | 113 | 400 |
| 8 | 30 | 135 | 32 | 49 | 150 |
| 9 | 33 | 177 | 39 | 238 | 240 |
| 10 | 43 | 375 | 47 | 142 | 322 |
| 11 | 33 | 300 | 37 | 129 | 321 |
| mean value | 33.5 | 247.4 | 37.6 | 114.0 | 295.5 |
| standard deviation | 3.9 | 87.2 | 4.4 | 53.6 | 80.2 |
| t-Test | | 0.0000 | 0.0000 | 0.0004 | 0.0000 |
| versus control | | | | | |
| versus others | | Hep v NCT | NCT v Hep + NCT | 0.01 v 0.025 | Hep v Hep + NCT |
| | | 0.0000 | 0.0000 | 0.0006 | 0.0445 |

TABLE 3
Prothrombin time (PT) (%)

| Subject no. | Control | Heparin | 0.01% NCT | 0.025% NCT | 0.01% NCT + Heparin |
|---|---|---|---|---|---|
| 1 | 96 | 89 | 86 | 25 | 68 |
| 2 | 86 | 69 | 72 | 16 | 61 |
| 3 | 89 | 78 | 77 | 36 | 72 |
| 4 | 83 | 68 | 74 | 24 | 60 |
| 5 | 84 | 72 | 79 | 32 | 65 |
| 6 | 82 | 66 | 70 | 28 | 58 |
| 7 | 72 | 58 | 59 | 20 | 52 |
| 8 | 102 | 89 | 95 | 50 | 84 |
| 9 | 73 | 62 | 62 | 10 | 51 |
| 10 | 91 | 74 | 88 | 21 | 69 |
| 11 | 67 | 54 | 61 | 16 | 48 |
| mean value | 84.1 | 70.8 | 74.8 | 25.3 | 62.5 |
| standard deviation | 10.5 | 11.3 | 11.7 | 11.1 | 10.5 |
| t-Test | | 0.0000 | 0.0000 | 0.0000 | 0.0000 |
| versus control | | | | | |
| versus others | | Hep v NCT | NCT v Hep + NCT | 0.01 v 0.025 | Hep v Hep + NCT |
| | | 0.0191 | 0.0000 | 0.0000 | 0.0001 |

TABLE 4
International normalized ratio (INR)

| Subject no. | Control | Heparin | 0.01% NCT | 0.025% NCT | 0.01% NCT + Heparin |
|---|---|---|---|---|---|
| 1 | 1.1 | 1.1 | 1.1 | 2.8 | 1.2 |
| 2 | 1.1 | 1.2 | 1.2 | 4.4 | 1.3 |
| 3 | 1.1 | 1.2 | 1.2 | 2 | 1.2 |
| 4 | 1.1 | 1.2 | 1.2 | 2.9 | 1.3 |
| 5 | 1.1 | 1.2 | 1.2 | 2.3 | 1.3 |
| 6 | 1.1 | 1.2 | 1.2 | 2.6 | 1.4 |
| 7 | 1.2 | 1.3 | 1.3 | 3.3 | 1.4 |
| 8 | 1 | 1.1 | 1.1 | 1.5 | 1.1 |
| 9 | 1.2 | 1.3 | 1.3 | 6 | 1.4 |
| 10 | 1.1 | 1.2 | 1.1 | 3.3 | 1.2 |
| 11 | 1.2 | 1.4 | 1.3 | 4.2 | 1.5 |
| mean value | 1.1 | 1.2 | 1.2 | 3.2 | 1.3 |
| standard deviation | 0.1 | 0.1 | 0.1 | 1.3 | 0.1 |
| t-Test | | 0.0000 | 0.0001 | 0.0002 | 0.0000 |
| versus control | | | | | |
| versus others | | Hep v NCT | NCT v Hep + NCT | 0.01 v 0.025 | Hep v Hep + NCT |
| | | 0.1669 | 0.0004 | 0.0003 | 0.0011 |

TABLE 5
Fibrinogen (mg/dl)

| Subject no. | Control | Heparin | 0.01% NCT | 0.025% NCT | 0.01% NCT + Heparin |
|---|---|---|---|---|---|
| 1 | 274 | 379 | 226 | 40 | 224 |
| 2 | 315 | 309 | 237 | 40 | 232 |
| 3 | 266 | 259 | 224 | 40 | 226 |
| 4 | 285 | 283 | 230 | 40 | 235 |
| 5 | 247 | 249 | 215 | 40 | 205 |
| 6 | 211 | 214 | 170 | 40 | 167 |
| 7 | 160 | 155 | 112 | 40 | 118 |
| 8 | 318 | 313 | 240 | 40 | 244 |
| 9 | 157 | 153 | 87 | 40 | 87 |
| 10 | 235 | 234 | 194 | 40 | 196 |
| 11 | 243 | 236 | 178 | 40 | 184 |
| mean value | 246.5 | 253.1 | 192.1 | 40.0 | 192.5 |
| standard deviation | 54.1 | 67.3 | 51.6 | 0.0 | 50.7 |
| t-Test | | 0.2587 | 0.0000 | 0.0000 | 0.0000 |
| versus control | | | | | |
| versus others | | Hep v NCT | NCT v Hep + NCT | 0.01 v 0.025 | Hep v Hep + NCT |
| | | 0.0001 | 0.7721 | 0.0000 | 0.0002 |

TABLE 6
Thrombin time (TT) (sec)

| Subject no. | Control | Heparin | 0.01% NCT | 0.025% NCT | 1% NCT + Heparin |
|---|---|---|---|---|---|
| 1 | 19 | 400 | 23 | 55 | 400 |
| 2 | 21 | 400 | 23 | 48 | 400 |
| 3 | 18 | 400 | 22 | 40 | 400 |
| 4 | 19 | 400 | 23 | 45 | 400 |
| 5 | 17 | 400 | 22 | 35 | 400 |
| 8 | 19 | 400 | 22 | 44 | 400 |
| 7 | 21 | 400 | 24 | 44 | 400 |
| 8 | 19 | 400 | 22 | 39 | 400 |
| 9 | 23 | 400 | 32 | 59 | 400 |
| 10 | 19 | 400 | 22 | 43 | 400 |
| 11 | 19 | 400 | 24 | 52 | 400 |
| mean value | 19.5 | 400.0 | 23.5 | 45.8 | 400.0 |
| standard deviation | 1.6 | 0.0 | 2.9 | 7.2 | 0.0 |
| t-Test | | 0.0000 | 0.0000 | 0.0000 | 0.0000 |
| versus control | | | | | |
| versus others | | Hep v NCT | NCT v Hep + NCT | 0.01 v 0.025 | Hep v Hep + NCT |
| | | 0.0000 | 0.0000 | 0.0000 | #DIV/0I |

TABLE 7
Antithrombin III (%)

| Subject no. | Control | Heparin | 0.01% NCT | 0.025% NCT | 1% NCT + Heparn |
|---|---|---|---|---|---|
| 1 | 94 | 113 | 94 | 87 | 93 |
| 2 | 77 | 75 | 75 | 73 | 73 |
| 3 | 97 | 94 | 95 | 94 | 92 |
| 4 | 80 | 81 | 80 | 78 | 80 |
| 5 | 84 | 83 | 84 | 79 | 82 |
| 6 | 96 | 93 | 92 | 90 | 91 |
| 7 | 81 | 79 | 79 | 81 | 73 |
| 8 | 99 | 99 | 101 | 101 | 102 |
| 9 | 85 | 83 | 85 | 79 | 78 |
| 10 | 82 | 81 | 82 | 79 | 84 |
| 11 | 87 | 88 | 81 | 80 | 82 |
| mean value | 87.5 | 88.1 | 86.2 | 83.7 | 84.5 |
| standard deviation | 7.7 | 11.0 | 8.1 | 8.3 | 9.0 |
| t-Test | | 0.3713 | 0.0889 | 0.0017 | 0.0114 |
| versus control | | | | | |
| versus others | | Hep v NCT | NCT v Hep + NCT | 0.01 v 0.025 | Hep v Hep + NCT |
| | | 0.3303 | 0.0849 | 0.0120 | 0.0893 |

TABLE 8
D-Dimer (μg/l)

| Subject no. | Control | Heparin | 0.01% NCT | 0.025% NCT | 1% NCT + Heparin |
|---|---|---|---|---|---|
| 1 | 243 | 285 | 241 | 227 | 248 |
| 2 | 162 | 159 | 155 | 143 | 162 |
| 3 | 137 | 150 | 132 | 126 | 136 |
| 4 | 197 | 203 | 191 | 172 | 195 |
| 5 | 144 | 157 | 149 | 132 | 150 |
| 6 | 213 | 217 | 209 | 200 | 213 |
| 7 | 199 | 199 | 195 | 179 | 199 |
| 8 | 351 | 353 | 340 | 316 | 350 |
| 9 | 144 | 146 | 143 | 122 | 143 |
| 10 | 68 | 76 | 50 | 50 | 50 |
| 11 | 186 | 183 | 174 | 153 | 177 |
| mean value | 185.8 | 193.5 | 179.9 | 165.5 | 184.6 |
| standard deviation | 72.1 | 74.2 | 72.6 | 68.1 | 73.7 |
| t-Test | | 0.0367 | 0.0096 | 0.0000 | 0.2181 |
| versus control | | | | | |
| versus others | | Hep v NCT | NCT v Hep + NCT | 0.01 v 0.025 | Hep v Hep + NCT |
| | | 0.0042 | 0.0004 | 0.0001 | 0.0247 |

### REFERENCES

1. Dakin H. D., “The behaviour of hypochlorites on intravenous injection and their action on blood serum,” BMJ 1916: 852-54.
2a. Nagl M. & Gottardi W., “In vitro experiments on the bactericidal action of N-chlorotaurine,” HYG MED 1992; 17: 431-39.
2. Nagl M. & Gottardi W., “Enhancement of the bactericidal efficacy of N-chlorotaurine by inflammation samples and selected N—H compounds,” HYG MED 1996; 21: 597-605.
3. Nagl M & Gottardi W., “Rapid killing of Mycobacterium terrae by N-chlorotaurine in presence of ammonium is caused by the reaction product monochloramine,” J PHARM PHARMACOL 1998; 50: 1317-20.
4. Gottardi W. & Nagl M., “Chemical properties of N-chlorotaurine sodium, a key compound in the human defence system” ARCH PHARM MED CHEM 2002; 335: 411-21.
5. van Gelder N. M. and Bowers R. J., “Synthesis and characterization of N,N-dichlorinated amino acids: taurine, homotaurine, GABA and L-leucine,” NEUROCHEMICAL RESEARCH 2001; 26(6): 575-78
6. Gottardi W. et al., “N,N-Dichlorotaurine: chemical and bactericidal properties,” ARCH PHARM MED CHEM 2005; 338: 473-83.
7. Gottardi W. et al., “N-alorotaurine and ammonium chloride: An antiseptic preparation with strong bactericidal activity,” INT. J. PHARMACEUT. 2007; 335(1-2): 32-40.
8. Grisham et al., “Chlorination of endogenous amines by isolated neutrophils,” JOURNAL OF BIOLOGICAL CHEMISTRY 1984; 259: 10404-13
9. Weiss, “Tissue destruction by neutrophils,” NEW ENGLAND JOURNAL OF MEDICINE 1989; 320: 365-76.
10. Kontny et al., “Taurine chloramine inhibition of cell proliferation and cytokine production by rheumatoid arthritis fibroblast-like synoviocytes,” ARTHRITIS AND RHEUMATISM 1999; 42:2552-60.
11. Park et al., “Regulation of nitric oxide induced by mycobacterial lipoarabinomannan in murine macrophages: effects of interferon-beta and taurine-chloramine,” INTERNATIONAL JOURNAL OF LEPROSY AND OTHER MYCOBACTERIAL DISEASES 2000; 68: 444-51.
12. Marcinkiewicz, “Prostanoids and MPO-halide system products as a link between innate and adaptive immunity,” IMMUNOLOGY LETTERS 2003; 89: 187-91.
13. Nagl et al., “Tolerance of N-chlorotaurine, an endogenous antimicrobial agent, in the rabbit and human eye—a phase I clinical study,” JOURNAL OF OCULAR PHARMACOLOGY AND THERAPEUTICS 1998; 14: 283-90.
14. Romanowski et al., “N-Chlorotaurine is an Effective Antiviral Agent against Adenovirus In Vitro and in the Ad5/NZW Rabbit Ocular Model,” INVESTIGATIVE OPHTHALMOLOGY & VISUAL SCIENCE 2006; 47: 2021-26.
15. Neher et al., “Acute Otitis Externa: Efficacy and Tolerability of N-Chlorotaurine, a Novel Endogenous Antiseptic Agent,” LARYNGOSCOPE 2004; 114: 850-54.
16. Neher et al., “Tolerability of N-Chlorotaurine in Chronic Rhinosinusitis Applied via Yamik Catheter,” AURIS NASUS LARYNX 2005; 32: 359-64.
16. Nagl et al., “Tolerability and efficacy of N-chlorotaurine compared to chloramine T for treatment of chronic leg ulcers with purulent coating,” BR J DERMATOL 2003; 149: 590-97.
18. Unterberger et al., “N-chlorotaurine—local antibacterial therapy in urinary tract infections by omniresistant Pseudomonas aeruginosa,” 21 Annual Conference of the Arbeitsgemeinschaft Neurologische Intensivmedizin (ANIM), Innsbruck 2001.
19. Neher et al., “N-chlorotaurine, a novel endogenous antimicrobial agent: tolerability tested in a mouse model,” ARCH. OTOLARYNGOL. HEAD NECK SURG. 2001; 127: 530-33.
20. Neher et al., “Tolerability of N-chlorotaurine in the guinea pig middle ear—a pilot study using an improved application system,” ANNALS OF OTOLOGY, RHINOLOGY & LARYNGOLOGY 2004; 113: 76-81.
21. Verdrengh et al., “Inhibition of septic arthritis by local administration of taurine chloramine, a product of activated neutrophils,” JOURNAL OF RHEUMATOLOGY 2005; 32: 1513-17.
22. Nagl M et al., “Activity of N-chlorotaurine against herpes simplex- and adenoviruses,” ANTIVIRAL RES 1998; 38: 25-30.
23. Nagl M et al., “Impact of N-chlorotaurine on viability and production of secreted aspartyl proteinases of Candida spp.,” ANTIMICROB AGENTS CHEMOTHER 2002; 46: 1996-99.
24. Yazdanbakhsh M et al., “Killing of schistosomula by taurine chloramine and taurine bromamine,” AM J TROP MED HYG 1987; 37: 106-10.
25. Nagl et al., “Enhanced fungicidal activity of N-chlorotaurine in nasal secretion,” JOURNAL OF ANTIMICROBIAL CHEMOTHERAPY 2001; 47: 871-74.
26. Dychdala, “Chlorine and chlorine compounds,” in Disinfection, Sterilization and Preservation, 135-158, Lippincott Williams & Wilkins, Philadelphia (2001).
27. Stief et al., “Singlet Oxygen Inactivates Fibrinogen, Factor V, Factor VIII, Factor X, and Platelet Aggregation of Human Blood,” THROMBOSIS RESEARCH 2000; 97: 473-80.

What is claimed is:

1. A composition for antiseptic and anticoagulation treatment of a mammal, the composition comprising a therapeutically effective amount of one or more N-halogenated or N,N-dihalogenated amines.

2. The composition of claim 1, wherein the N-halogenated or N,N-dihalogenated amine is N-chloramine or N,N-dichloramine.

3. The composition of claim 1, wherein the N-halogenated or N,N-dihalogenated amine is a derivative of a protein, peptide, or amino acid.

4. The composition of claim 1 wherein the N-halogenated or N,N-dihalogenated amine is derived from an α-amino carbonic acid, a β-amino carbonic acid, an α-amino sulfonic acid, a β-amino sulfonic acid, or an aliphatic amine.

5. The composition of claim 1, wherein the N-halogenated or N,N-dihalogenated amine is N-chlorotaurine or N,N-dichlorotaurine.

6. The composition of claim 1, wherein the one or more N-halogenated or N,N-dihalogenated amines are in an aqueous solution at a concentration of about 0.001% to about 10%; preferably about 0.01% to about 5%; about 0.01% to about 2%; about 0.1% to about 2%; about 0.2% to about 1%; about 0.01% to about 0.035%; or about 0.01% to about 0.025%.

7. The composition of claim 1, wherein the N-halogenated or N,N-dihalogenated amine is in the form of a pharmaceutically acceptable salt; preferably an alkali salt; more preferably a sodium salt.

8. The composition of claim 1, further comprising an ammonium salt; preferably ammonium chloride.

9. The composition of claim 1, wherein the N-halogenated or N,N-dihalogenated amine is alkylated at a carbon.

10. The composition of claim 9, further comprising an ammonium salt.

11. A composition for preventing or treating a hospital-acquired infection or condition in a mammal comprising a therapeutically effective amount of one or more N-halogenated or N,N-dihalogenated amines.

12. The composition of claim 11, wherein the hospital-acquired infection or condition comprises at least one of a catheter-associated urinary tract infection; pressure ulcer; vascular catheter-associated infection; blood stream infection; surgical site infection; and mediastinitis after coronary artery bypass graft surgery.

13. A composition for preventing or treating an antibiotic-resistant pathogen in a mammal comprising a therapeutically effective amount of one or more N-halogenated or N,N-dihalogenated amines.

14. The composition of claim 13, wherein the antibiotic-resistant pathogen is a methicillin resistant Staphylococcus aureus (MRSA); vancomycin-resistant Enterococci (VRE); penicillin-resistant Enterococcus, linezolid-resistant Enterococcus, vancomycin-resistant Staphylococcus aureus (VRSA), and Acinetobacter baumannii.

15. The use of one or more N-halogenated or N,N-dihalogenated amines for the manufacture of a medicament for antiseptic and anticoagulation treatment of a mammal.

16. The use of claim 15, wherein the N-halogenated or N,N-dihalogenated amine is a N-mono or a N,N-dichloramine.

17. The use of claim 15, wherein the N-halogenated or N,N-dihalogenated amine is a derivative of a protein, peptide, or amino acid.

18. The use of claim 15, wherein the N-halogenated or N,N-dihalogenated amine is derived from an α-amino carbonic acid, a β-amino carbonic acid, an α-amino sulfonic acid, a β-amino sulfonic acid, or an aliphatic amine.

19. The use of claim 15 wherein the N-halogenated or N,N-dihalogenated amine is N-chlorotaurine or N,N-dichlorotaurine.

20. The use of claim 15, wherein the one or more N-halogenated or N,N-dihalogenated amines are in an aqueous solution at a concentration of about 0.001% to about 1 0%; preferably about 0.01% to about 5%; about 0.01% to about 2%; about 0.1% to about 2%; about 0.2% to about 1%; about 0.01% to about 0.035%; or about 0.01% to about 0.025%.

21. The use of claim 15, wherein the N-halogenated or N,N-dihalogenated amine is in the form of an alkali salt; preferably a sodium salt.

22. The use of claim 15, further comprising an ammonium salt; preferably ammonium chloride.

23. The use of claim 15, wherein the N-halogenated or N,N-dihalogenated amine is alkylated at a carbon.

24. The use of claim 23, further comprising an ammonium salt.

25. The use of one or more N-halogenated or N,N-dihalogenated amines for the manufacture of a medicament for preventing or treating a hospital-acquired infection or condition in a mammal, wherein the medicament comprises a therapeutically effective amount of one or more N-halogenated or N,N-dihalogenated amines.

26. The use of claim 25, wherein the hospital-acquired infection or condition comprises at least one of a catheter-associated urinary tract infection; pressure ulcer; vascular catheter-associated infection; blood stream infection; surgical site infection; and mediastinitis after coronary artery bypass graft surgery.

27. The use of one or more N-halogenated or N,N-dihalogenated amines for the manufacture of a medicament for preventing or treating an antibiotic-resistant pathogen in a mammal comprising a therapeutically effective amount of one or more N-halogenated or N,N-dihalogenated amines.

28. The use of claim 27, wherein the antibiotic-resistant pathogen is a methicillin resistant Staphylococcus aureus (MRSA); vancomycin-resistant Enterococci (VRE); penicillin-resistant Enterococcus, linezolid-resistant Enterococcus, vancomycin-resistant Staphylococcus aureus (VRSA), and Acinetobacter baumannii.

29. A pharmaceutical composition having antiseptic or anti-infective activity coupled with anticoagulant activity capable of at least one of treating or preventing a microbial infection, formation of blood platelet aggregates, formation of fibrin, thrombus formation, or embolus formation in a mammal, the composition comprising a therapeutically effective amount of one or more N-halogenated or N,N-dihalogenated amines.

30. A medical device comprising a therapeutically effective amount of one or more N-halogenated or N,N-dihalogenated amines, wherein the one or more N-halogenated or N,N-dihalogenated amines are incorporated into or coated onto the device.

31. The medical device of claim 30, wherein the device is adapted for at least one of implantation into a body of a mammal; contacting a skin or bodily organ, and penetration of a skin or bodily organ.

32. The medical device of claim 30, wherein the device is adapted for at least one of blood collection, blood circulation, and blood storage.

33. The medical device of claim 30, wherein the device is a catheter, blood dialysis machine, blood collection syringe, tube, blood line, urinary tract catheter, central line catheter, central venous catheter, IV drip unit, implantable catheter, shunt, or stent.

34. A method of inhibiting infection and coagulation in a mammal comprising treating a mammal in need of such treatment with a therapeutically effective amount of one or more N-halogenated or N,N-dihalogenated amines to inhibit infection and coagulation.

35. The method of claim 34, wherein the therapeutically effective amount of one or more N-halogenated or N,N-dihalogenated amines is incorporated into or coated onto a medical device that is introduced into the mammal.

36. The method of claim 34, wherein inhibiting infection and coagulation comprises at least one of prevention of infection and blood coagulation within a catheter; treating a urinary disorder; performing hemodialysis; providing an artificial shunt or joint; treating or preventing a myocardial infarction, unstable angina, stroke, restenosis, deep vein thrombosis, disseminated intravascular coagulation caused by trauma, sepsis or tumor metastasis, cardiopulmonary bypass surgery, hypercoagulability during chemotherapy, or fibrin formation in the eye; and facilitating wound healing.

37. A method for preventing or treating a hospital-acquired infection or condition, wherein a mammal in need of such treatment is provided with a therapeutically effective amount of one or more N-halogenated or N,N-dihalogenated amines.

38. The method of claim 37, wherein the hospital-acquired infection or condition comprises at least one of a catheter-associated urinary tract infection; pressure ulcer; vascular catheter-associated infection; blood stream infection; surgical site infection; and mediastinitis after coronary artery bypass graft surgery.

39. A method for preventing or treating a antibiotic-resistant pathogen, wherein a mammal in need of such treatment is provided with a therapeutically effective amount of one or more N-halogenated or N,N-dihalogenated amines.

40. The method of claim 39, wherein the antibiotic-resistant pathogen is a methicillin resistant Staphylococcus aureus (MRSA); vancomycin-resistant Enterococci (VRE); penicillin-resistant Enterococcus, linezolid-resistant Enterococcus, vancomycin-resistant Staphylococcus aureus (VRSA), and Acinetobacter baumanii.

41. The method of any of claims 34-40, wherein the mammal is a human.

42. A composition for antiseptic and anticoagulation treatment of a mammal, the composition comprising a therapeutically effective amount of one or more N-halogenated or N,N-dihalogenated amines.

43. The composition of claim 42, wherein the N-halogenated or N,N-dihalogenated amine is N-chloramine or N,N-dichloramine.

44. The composition of claim 42, wherein the one or more N-halogenated or N,N-dihalogenated amines are in an aqueous solution at a concentration of about 0.001% to about 10%.

45. The composition of claim 42, wherein the N-halogenated or N,N-dihalogenated amine is in the form of a pharmaceutically acceptable salt.

46. The composition of claim 42, wherein the N-halogenated or N,N-dihalogenated amine is alkylated at a carbon.

47. The composition of claim 42, wherein the N-halogenated or N,N-dihalogenated amine is N,N-dichloro-2,2-dimethyl taurine.

48. A composition for preventing or treating an antibiotic-resistant pathogen in a mammal comprising a therapeutically effective amount of one or more N-halogenated or N,N-dihalogenated amines.

49. The composition of claim 48, wherein the antibiotic-resistant pathogen is a methicillin resistant Staphylococcus aureus (MRSA); vancomycin-resistant Enterococci (VRE); penicillin-resistant Enterococcus, linezolid-resistant Enterococcus, vancomycin-resistant Staphylococcus aureus (VRSA), and Acinetobacter baumannii.

50. The composition of claim 48, wherein the composition further comprises a therapeutically effective amount of an anticoagulant, an anti-platelet agent or a thrombolytic agent.

51. The composition of claim 48, wherein the anticoagulant is heparin.

52. A method for the treatment of antiseptic and anticoagulation of a mammal, the method comprising administrating a therapeutically effective amount of a composition comprising one or more N-halogenated or N,N-dihalogenated amines to a mammal in need thereof.

53. The method of claim 52, wherein the N-halogenated or N,N-dihalogenated amine is N-chloramine or N,N-dichloramine.

54. The method of claim 52, wherein the N-halogenated or N,N-dihalogenated amine is alkylated at a carbon.

55. The method of claim 52, wherein the N-halogenated or N,N-dihalogenated amine is N,N-dichloro-2,2-dimethyl taurine.

56. A pharmaceutical composition having antiseptic or anti-infective activity coupled with anticoagulant activity for treating or preventing of at least one of a microbial infection, formation of blood platelet aggregates, formation of fibrin, thrombus formation, or embolus formation in a mammal, the composition comprising a therapeutically effective amount of one or more N-halogenated or N,N-dihalogenated amines.
